(19) United States
(12) Patent Application Publication
(10) Pub. No.: US 2004/0260258 A1
(43) Pub. Date: Dec. 23, 2004

(54) Absorbent article having removable, edge-stiffening element

(76) Inventors: Adrienne Hall, Monmouth Junction, NJ (US); Claude Montcalm, Ile Bizard (CA); Jennifer L. Sturgeon, Long Valley, NJ (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/464,349

(22) Filed: Jun. 17, 2003

(51) Int. Cl. A61F 13/15; A61F 13/20

(52) U.S. Cl. 604/367; 604/385.01

(57) ABSTRACT

Methods of making an absorbent article and an absorbent article having a silhouette including opposed transverse edges and opposed longitudinally-extending edges that connect the transverse edges and a layered portion having at least one removable, edge-stiffening element substantially coterminal with a first terminal, edge-support point and substantially coterminal with a second terminal edge-support point, wherein the at least one removable edge-stiffening element is offset from an edge of the second longitudinally-extending edge, the first transverse edge, the second transverse edge, and combinations thereof, wherein the article has a flexural resistance less than about 50 grams.

### FIELD OF THE INVENTION

The invention relates to absorbent articles, and, in particular, to absorbent articles for use with undergarments or other clothing.

### BACKGROUND OF THE INVENTION

Disposable absorbent articles, such as, pantiliners, sanitary napkins, interlabial devices, adult incontinence devices and diapers are well known in the art. These articles typically have a fluid pervious body-facing side and a liquid impervious garment facing side. Additionally, such articles may include other functional components, such as, positioning adhesive, to reduce the tendency of the article to shift position relative to wearer's body, in use. The positioning adhesive is typically protected by release paper that is removed just before a wearer first positions the article adjacent to the wearer's body.

Anatomical adaptation of an absorbent article to the wearer's body may increase comfort to the wearer as well as reduce leakage of bodily exudates from the article. One approach to improve anatomical adaptation of the article is to reduce the flexural resistance (e.g., improve the flexibility of the article or otherwise increase the ability of the article to follow the contours of a surface that the article is placed against), the thickness, or both of the article.

Unfortunately, various problems may result related to the pursuit of absorbent articles having reduced flexural resistance, thickness, or both. For example, in a typical disposable absorbent article manufacturing process, individual articles that have been assembled must be grouped together and placed in an appropriate package, such as, a box, carton, or plastic film. In order to facilitate placement of individual articles into the package, it is common practice to feed the assembled absorbent articles into a stacking device. The absorbent articles are fed into the stacking device in a manner such that the absorbent articles are supported on a longitudinal edge of the article. This is problematic for articles having reduced flexural resistance, since the stiffness of the edge of the article may not support the weight of the article. As a result, such articles tend to droop or fall in the stacking device, thereby requiring the line to be shut down or resulting in errors in the number of articles placed in the package.

One solution to the problem of poor stacking in low flexural resistance absorbent articles is to have release paper extend across the entire article. The release paper imparts stiffness to the edge of the article and thereby improves stacking performance. Unfortunately, by extending the release paper across the entire article, the release paper may be difficult to remove, which results in increased wearer frustration and inconvenience. Therefore, a need exists for an easy-to use, as well as easy-to-manufacture and package absorbent article that has reduced or low flexural resistance.

The absorbent article of the present invention is advantageous in that the absorbent article includes a removable edge stiffening element, making the article capable of being supported on a longitudinally-extending edge. This facilitates stacking, counting, and packaging of the articles. Furthermore, the removable edge stiffening element is configured for easy removal prior to use of the article. In at least one embodiment of the invention, removal of the edge stiffening element reveals a securing element to facilitate securing to a wearer's clothing.

### SUMMARY OF THE INVENTION

The present invention is directed to an absorbent article comprising, consisting essentially of or consisting of a silhouette including a first transverse edge, a second transverse edge, wherein the second transverse edge being in opposite relation to the first transverse edge, and a first longitudinally-extending edge having a first terminal edge-support point and a second terminal edge-support point and a second longitudinally extending edge being in opposite relation to the first longitudinally, wherein the first longitudinally extending edge and the second longitudinally extending edge connect the first transverse edge and the second transverse edge; and a layered portion having at least one removable, edge-stiffening element substantially coterminal with the first terminal edge-support point and substantially coterminal with the second terminal edge-support point, wherein the at least one removable edge-stiffening element is offset from an edge selected from the group consisting of the second longitudinally-extending edge, the first transverse edge, the second transverse edge, and combinations thereof, wherein the article has a flexural resistance less than about 50 grams.

In another aspect of the invention, a method of forming an absorbent article, comprises, consisting essentially of or consists of providing a web of material, the web of material having a first edge, a second edge opposed to the first edge, and a directional center between the first edge and the second edge; superimposing a sheet of edge stiffening material on the web of material such that a first edge of the sheet of edge stiffening material is registered between the first edge of the web of material and the second edge of the web of material, wherein the superposition of the sheet of edge stiffening material and the web of material defines a region of overlap and a region free of removable, edge stiffening material, the region of overlap being adjacent to the region of removable, edge stiffening material; and severing both the web of material and the web of removable, edge stiffening material superimposed thereupon along a enclosed trace, wherein the enclosed trace defines a silhouette that includes a first transverse edge, a second transverse edge in opposite relation to the first transverse edge, and a first longitudinally-extending edge having a first terminal edge-support point and a second terminal edge-support point, a second longitudinally extending edge in opposite relation to the first longitudinally-extending edge, the first longitudinally extending edge and the second longitudinally extending edge connecting the first transverse edge and the second transverse edge, wherein the first terminal edge-support point and a second terminal edge-support point are substantially within the region of overlap and an edge selected from the group consisting of the second longitudinally-extending edge, the first transverse edge, the second transverse edge, and combinations thereof extend into the region free of removable, edge stiffening material, wherein the article has a flexural resistance less than about 50 grams.

### BRIEF DESCRIPTION OF THE DRAWINGS

A more particular description of the invention that is briefly summarized above may be had by reference to the embodiments thereof that are illustrated in the appended drawings. It is to be so noted, however, that the appended drawings illustrate only typical embodiments of the invention and, therefore, are not to be considered limiting of its scope, for the invention may admit to other equally effective embodiments.

FIG. 1 shows a bottom plan view of an absorbent article consistent with an embodiment of the invention described herein;

FIG. 2 shows a bottom plan view of an embodiment of an absorbent article of the present invention;

FIG. 3 shows a bottom plan view of an embodiment of an absorbent article of the present invention;

FIG. 4a shows a bottom plan view of an embodiment of an absorbent article of the present invention;

FIG. 4b shows a bottom plan view of the absorbent article of FIG. 4a in an alternate configuration;

FIG. 5 shows a cross-sectional view of the absorbent article of FIG. 1 taken along line A-A;

FIG. 6 shows a bottom plan view of an embodiment of an absorbent article of the present invention;

FIG. 7 shows a bottom plan view of an embodiment of an absorbent article of the present invention; and

FIG. 8 shows a fragmented plan view.

### DETAILED DESCRIPTION OF THE INVENTION

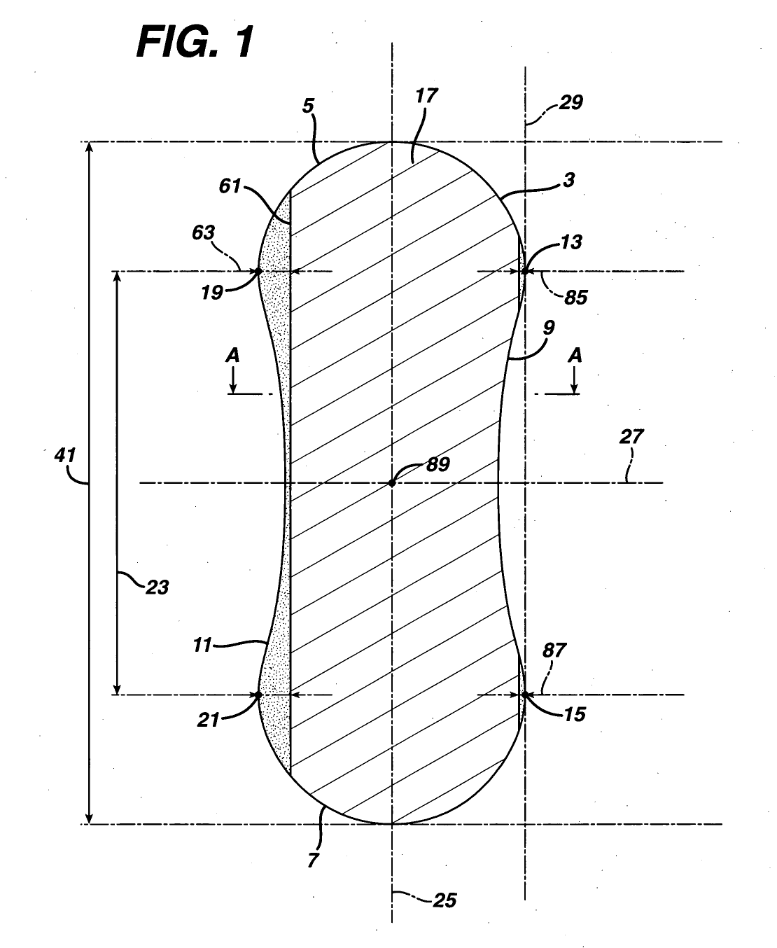

FIG. 1 depicts an absorbent article 1, which may be, for example, a pantiliner, a sanitary napkin, an interlabial device, an adult incontinence device, or a diaper, of the present invention. The absorbent article 1 has a silhouette 3 that generally defines the limits, edges, or boundaries of the absorbent article 1 (after any removable elements such as removable edge stiffening elements, release paper, and the like have been removed). The silhouette 3 includes a first transversely-extending edge 5 and a second transversely-extending edge 7 that is generally in opposite relation to the first transversely-extending edge 5. The silhouette 3 further includes a first longitudinally-extending edge 9 and a second longitudinally extending edge 11 that is generally in opposite relation to the first longitudinally-extending edge 9. The first longitudinally-extending edge 9 and the second longitudinally extending edge 11 connect the first transversely-extending edge 5 and the second transversely-extending edge 7. The absorbent article may be further characterized by a longitudinally-extending centerline 25 and a transversely-extending centerline 27 (the centerlines 25, 27 are shown in phantom in FIG. 1) that are centrally disposed, as shown in FIG. 1. The centerlines 25, 27 generally pass through a centerpoint 89 of the absorbent article and are orthogonal to one another. The centerlines 25, 27 may each divide symmetrical halves of the absorbent article 1. In this embodiment of the invention, the longitudinally-extending centerline 25 and the transversely-extending centerline 27 are lines of symmetry. While FIG. 1 depicts the absorbent article 1 as longitudinally symmetric and transversely symmetric, such symmetry is not required and the absorbent article may have only longitudinal symmetry, transverse symmetry, or neither.

The first longitudinally-extending edge 9 has a first terminal edge-support point 13 and a second terminal edge-support point 15. The first terminal edge-support point 13 and the second terminal edge-support point 15 are points on the silhouette 3 that are capable of serving as points of mechanical self support if the absorbent article 1 was positioned to stand upon its first longitudinally-extending edge 9 and the absorbent article 1 had sufficient stiffness or rigidity to be so supported. Thus, in this scenario, compression forces are imposed upon the absorbent article across line 29 (shown in phantom in FIG. 1) and the absorbent article would prefer to rest upon two points of tangency that the line 29 forms with the absorbent article 1. These two points of tangency are the first terminal edge-support point 13 and the second terminal edge-support point 15.

In this embodiment, the first longitudinally terminal edge-support point 13 and the second terminal edge-support point 15 may be defined as the points that fulfill two criteria: (1) they are the points most laterally outward from the longitudinally-extending centerline 25 and (2) of all the points that are most laterally outward from the longitudinally-extending centerline 25, they are the points on either side of the transversely-extending centerline 27 that are greatest in distance from the transversely-extending centerline 27.

Analogous edge points 19, 21 are present on the silhouette 3 and are located in a similar manner as described above for the first longitudinally terminal edge-support point 13 and the second terminal edge-support point 15. The edge points 19, 21 are located in a manner similar to the manner described above for the terminal edge support points 13, 15, except that the absorbent article is turned to stand upon the second longitudinally-extending side 11. Points 13, 15, 19, 21 generally define the limits of the first transversely-extending edge 5, the second transversely-extending edge 7, the first longitudinally-extending edge 9, and the second longitudinally extending edge 11. The first longitudinally-extending edge is defined by all the points on the silhouette 3 along the shortest line or arc between point 13 and point 15, inclusive of point 13 and point 15. The second longitudinally-extending edge is thus defined by all the points on the silhouette 3 along the shortest line or arc between point 19 and point 21, inclusive of point 19 and point 21. The first transversely-extending edge 5 is defined by all the points on the silhouette 3 that are between point 13 and point 19, inclusive of point 13 and point 19. The second transversely-extending edge 7 is defined by all the points on the silhouette 3 along the shortest line or arc between point 15 and point 21, inclusive of point 15 and point 21.

The silhouette 3 of the absorbent article 1 has a length 41 that is the longitudinal span of the silhouette 3 (i.e., the greatest distance measured parallel to the longitudinally-extending centerline 25 that separates two points on the silhouette 3). The first longitudinally-extending edge 9 and the second longitudinally-extending edge 11 are spaced apart at a longitudinal distance 23 that is a fraction of the length 41. A ratio of the distance 23 to the length 41 may be less than about 0.9. In one embodiment of the invention, the ratio of the distance 23 to the length 41 is between about 0.4 and about 0.7. Note that as the ratio of the distance 23 to the length 41 decreases, generally the greater is the need for edge stiffening in the absorbent article 1. This is because for low ratios of the distance 23 to the length 41, generally the more mass of the article 1 is concentrated about the edges 5, 7. As such, there are greater forces on the article to fall or droop when the absorbent article is placed upon the first longitudinally-extending edge 9.

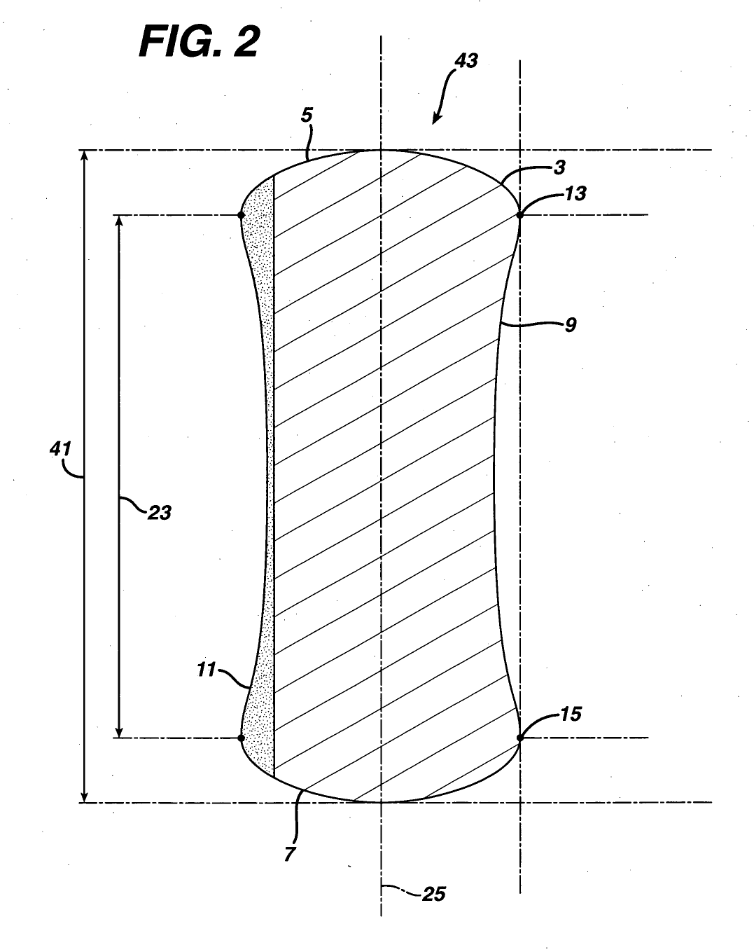

FIG. 2 depicts an alternative embodiment of the absorbent article 1 of the present invention. In particular, the absorbent article 1 has a silhouette 3 with the ratio of the distance 23 to the length 41 that is greater than the absorbent article 1 of FIG. 1. The first longitudinally-extending edge 9, as shown in FIGS. 1 and 2, is convex with respect to the longitudinally-extending centerline 25 (i.e., the first longitudinally-extending edge 9 is increasingly further from the longitudinally-extending centerline 25 as one moves away from the transversely-extending centerline 27.

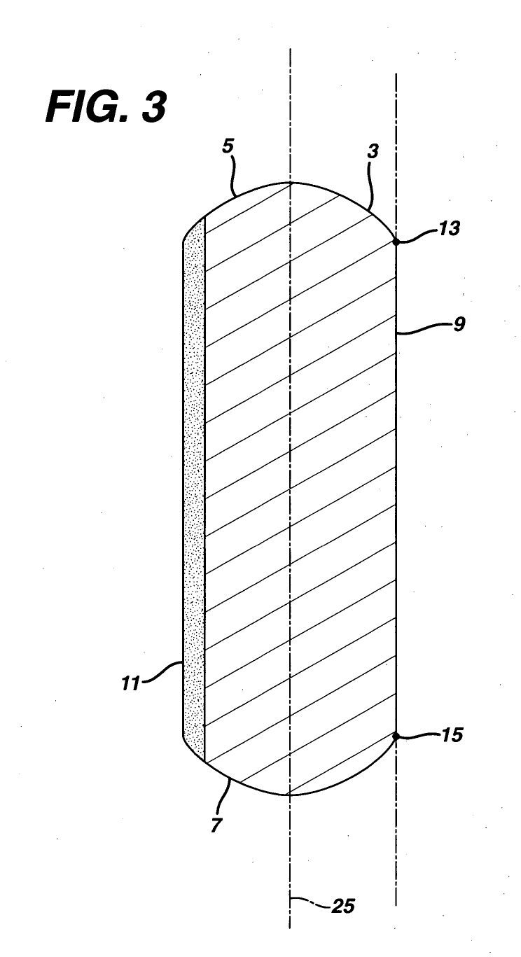

The absorbent article 1 depicted in FIG. 3 has a first longitudinally-extending edge 9 that is linear. It is generally preferred that the first longitudinally-extending edge 9 of the absorbent article 1 is either linear or convex with respect to the longitudinally-extending centerline 25 (i.e., the first longitudinally-extending edge 9 is preferably. “non-concave” with respect to the longitudinally-extending centerline 25). As such, the absorbent article 1 is capable of readily being supported along the first longitudinally-extending edge 9 without drooping or falling.

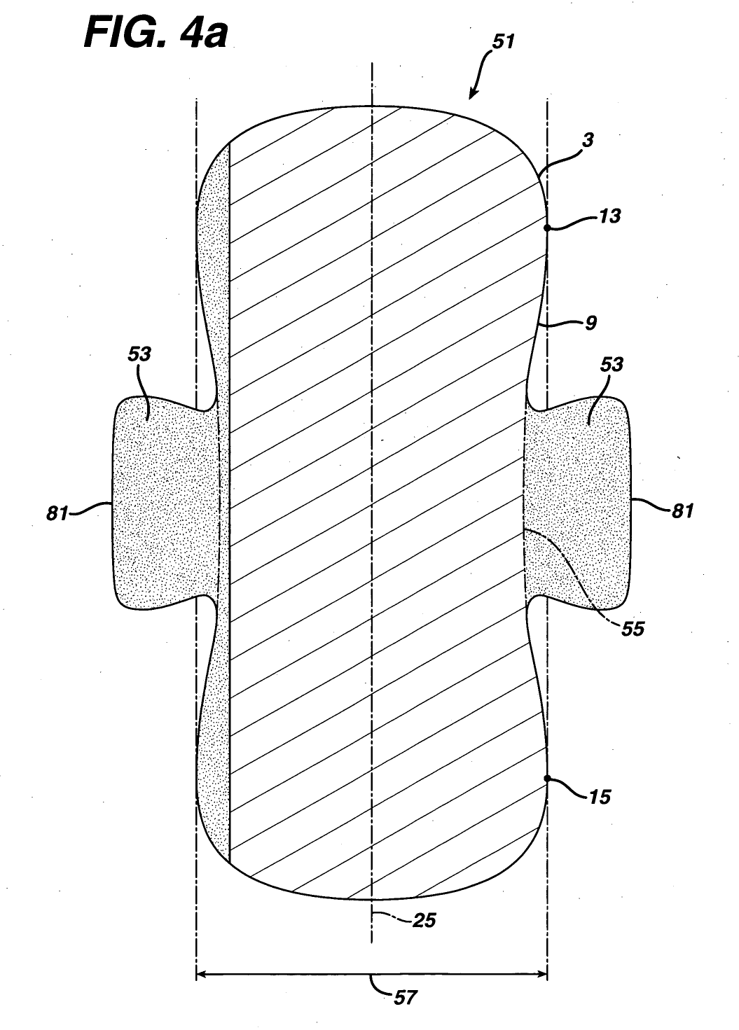

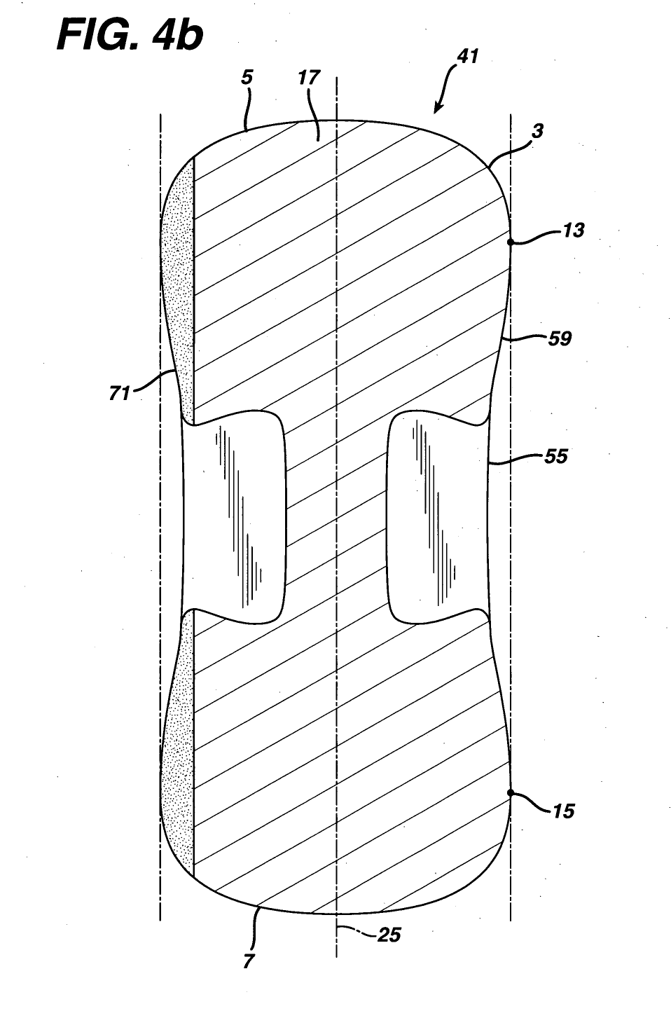

Referring to FIGS. 4a and 4b, an alternative embodiment of the invention, an absorbent article 51 has a silhouette 3 that is such that the first longitudinally extending edge 9 is partially concave with respect to the longitudinally-extending centerline 25. In particular, the absorbent article 51 includes wings or flaps 53. The flaps 53 each extend outward from respective proximal edge that may be coincident with the axis 55 to respective distal ends 81. The flaps 53 are adapted to fold about an axis 55 around an edge of a user's undergarment. Absorbent articles having flaps, and their use in sanitary protection articles, are described in U.S. Pat. Nos. 4,687,478; 4,589,876; 4,900,320; and U.S. Pat. No. 4,608,047. The disclosures of these documents are incorporated herein by reference in their entirety. As disclosed in the above documents, the flaps 53 are generally speaking flexible and have variable shapes. For example, the flaps 53 may be rounded, rectangular, curvilinear, etc. The flaps may be regular or irregular, symmetric or asymmetric in shape.

Upon folding the flaps 53 inwardly, as depicted in FIG. 4b, it can be readily seen that the absorbent article 51 includes the folded, non-concave, first longitudinally-extending edge 59, a second folded, non-concave, longitudinally-extending edge 71, as well as the first transverse edge 5 and the second transverse edge 7, which are similar to the absorbent article 1 shown in FIGS. 1, 2 and 3. As shown in FIG. 4a, the absorbent article 51 has a width 57 (the greatest transverse extent after folding the flaps 53). After folding of the flaps 53, the absorbent article 51 may be stacked or otherwise supported upon the folded, first longitudinally-extending edge 59 While FIGS. 1, 2, and 3 show absorbent articles having various shapes of the silhouette 3, other shapes, both symmetrical and asymmetrical are also contemplated, e.g., absorbent articles having parallel longitudinal edges, dog bone, peanut-shaped, shaped to fit thong garments, and the like. The overall dimensions of the absorbent article 1, 51 of the present invention is not critical. For example, the length of the absorbent article 1, 51 may from about 125 mm to about 315 mm. The width of the absorbent article may be from about 5 mm to about 115 mm.

Construction of the Absorbent Article

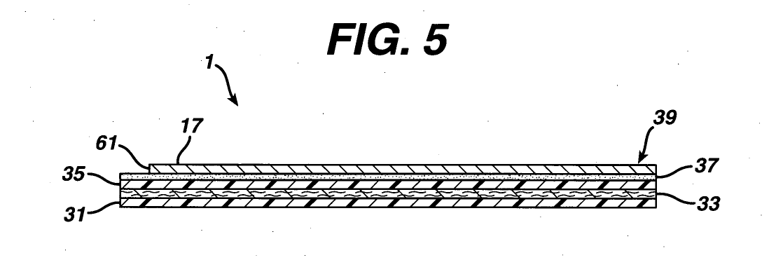

Referring to FIG. 1 as well as FIG. 5, the absorbent article 1, 51 has a layered structure 39. The layered structure 39 includes one or more functional material layers, such as, for example, a body-faceable, fluid-pervious cover layer 31 and a garment faceable, liquid-impervious barrier layer 35. In another embodiment of the invention, the absorbent article 1 additionally includes an absorbent system 33 intermediate the cover layer 31 and the barrier layer 35. The absorbent system 33 may include one or more layers, such as, a transfer layer useful for wicking liquids and an absorbent core useful for retaining liquids. While the layered structure 39 in FIG. 5 is depicted as having a plurality of discrete layers, one or more of these layers may be unitized into one or more multi-functional, unitized layers.

The cover layer 31 is generally compliant, soft feeling, and non-irritating to the user's skin. It can be made from any of the materials conventional for this type of use. The cover layer 31 generally functions to transport fluid away from the wearer into the absorbent article 1. In this manner, fluid and moisture are removed from contacting the wearer, thus making the wearer feel dry and comfortable. Non-limiting examples of suitable materials that can be used as the cover layer 31 are woven and nonwoven fabrics formed from polyester, polypropylene, nylon, and/or rayon fibers or the cover layer 31 may be an apertured thermo-plastic film and formed films. The cover layer 31 may optionally be treated with surfactant to manipulate the hydrophobicity/hydrophilicty thereof to facilitate optimal fluid transport properties. The fibers or other materials that make up the cover layer 31 should not collapse or lose their resiliency when subjected to body fluid. The fibers may be oriented by a carding process and thermally bonded via embossing. The thickness of the cover layer 31 may vary from, about 0.025 mm to about 5 mm, depending on the material chosen. The weight of the body facing layer material should be between about 5 to about 150 grams per square meter (gsm).

In one embodiment of the invention, the cover layer 31 includes a spunlace non-woven. In particular, the spunlace material may be made from about 0 to about 100% rayon and from about 0 to about 100% polyester. The spunlace material may also be made from about 10 to about 65% rayon and from about 35 to about 90% polyester may be used. Optionally, the material used for the body-facing layer may include binders, such as thermoplastic binder fibers and latex binders. In addition to transporting fluid, the cover layer 31 may also absorb and/or retain fluid as well.

The barrier layer 35 is impervious to liquids and, thus, prevents bodily fluids from soiling the clothing of the user. Suitable materials that may be incorporated into the barrier layer 35 include, for example, embossed or non-embossed polyethylene films, microporous films, and laminated tissue, among other materials. In one embodiment of the invention, the barrier layer 35 layer is a thin, flexible, fluid impervious material, such as, a polymeric film, e.g., polyethylene, polypropylene, or cellophane, or a normally liquid pervious material that has been treated to be impervious, such as, impregnated fluid repellent paper or non-woven material, including non-woven fabric material, or a flexible foam, such as polyurethane or cross-linked polyethylene. The thickness of the barrier layer when formed from a polymeric film may be about 0.025 mm to about 0.051 mm.

An absorbent system 33 may be positioned intermediate the cover layer 31 and the barrier layer 35. The absorbent system 33 provides a means for absorbing bodily fluid. Bodily fluid moving inward or “down” from the cover layer 31 is conveyed to the absorbent system 33 which retains the bulk of the fluid until the absorbent article 1, 51 is discarded. The absorbent system 33 may include two separate layers such as a transfer or acquisition layer and an absorbent core. The transfer layer and the absorbent core may be discrete layers or may be unitized.

The transfer layer or distribution layer, if present, is generally positioned beneath the cover layer 31, and the transfer layer usually directly contacts the absorbent core. The transfer layer provides a means of receiving body fluid from the fluid-pervious cover layer 31 and holding it until the absorbent core has an opportunity to absorb it. The transfer layer is, preferably, more dense than the cover layer 31 and has a larger proportion of smaller pores than does the cover layer 31. These attributes allow the transfer layer to contain body fluid and hold it away from the outer side of the cover layer 31, thereby preventing the fluid from re-wetting the cover layer 31 and its outer surface. However, the transfer layer is preferably not so dense as to prevent the passage of the fluid through the transfer layer and into the underlying absorbent core.

The transfer layer may include various materials, including, for example, fibrous webs, resilient foams, and the like. The transfer layer may include cellulose fibers such as from wood pulp, single component or bicomponent fibers that include thermoplastic materials (such as polyester, polypropylene, polyethylene, among others) in fiber or other forms, rayon, organic binders (such as copolymers of vinyl, acrylic and/or other monomers that may be coated onto thermoplastic fibers or otherwise incorporated into the transfer layer) among other materials known to the art. The transfer layer may, for example, have a basis weight in a range from about 40 gsm to about 120 gsm, a thickness in a range from about 0.5 mm to about 4 mm, a density in a range from about 0.03 g/cc to about 0.15 g/cc.

The mass of materials making up the transfer layer may be absorbent, although the materials themselves are not absorbent. Thus, transfer layers that are made of hydrophobic, nonabsorbent fibers may be able to accept large volumes of fluid into interfiber void spaces while the fibers themselves do not absorb any significant quantities of fluid. Likewise, open-celled foam structures that are made from nonabsorbent materials may also absorb fluid into the cells of the foam. The walls of the cells, however, do not absorb any fluid. The cumulative spaces within the transfer layer, i.e., the interfiber void spaces in the fibrous transfer layer or the open cells in the foam transfer layer, function much like a container to hold fluid.

Typically, transfer layer fibrous webs are made of resilient, nonabsorbent materials to provide void volume and to allow for free movement of fluid through the structure. Transfer layers that are made from webs of mostly absorbent fibers absorb the fluid as it enters the structure and do not distribute it throughout the rest of the structure as efficiently as webs containing non-absorbent materials.

The absorbent core, which may be positioned underneath the optional transfer layer, has a high capacity for absorbing liquids and may be capable of maintaining the definition of the plurality of protrusions 23 and the plurality of channels 21 during the wearing of the sanitary napkin 1. Examples of material that may be used in the construction of the absorbent core include, for example, cellulosic fibers (preferably wood pulp, but cotton, flax and peat moss are acceptable), synthetic fibers, superabsorbent polymers (SAP) or superabsorbent fibers, as well organic binders or other materials described above as suitable for incorporation into the transfer layer, and other materials known to the art of manufacturing absorbent core materials. The relative proportion of these materials may be varied to achieve sufficient absorbency, compressibility, and processibility.

The absorbent core may be resin or thermal bonded, and flat or emboss calendared to achieve an appropriate strength. The density of the absorbent core is preferably in a range from about 0.08 g/cc to about 0.60 g/cc.

The combined thickness of all of the materials layers of the absorbent article (such as, the cover layer 31, the barrier layer 35 and the absorbent system 33), excluding any removable layers, may be in a range from about 0.5 mm to about 5 mm. In one embodiment of the invention, the combined thickness of all of the materials layers is in a range from about 0.6 mm to about 2.5 mm. In another embodiment of the invention, the combined thickness is in a range from about 0.6 mm to about 1.5 mm. Note that the cover layer 31, the barrier layer 35 and the absorbent system 33 may be secured to one another using any suitable construction adhesive now known or developed in future in the art of absorbent article manufacture.

Referring again to FIG. 5, the absorbent article 1 may include a securing element 37 for securing the absorbent article to the inside surface of the crotch portion of a garment. The securing element 37 may include, for example, adhesive, mechanical attachment devices, such as, clips, laces, ties, and interlocking devices, e.g., snaps, buttons, VELCRO (available from Velcro USA, Inc., Manchester, N.H.), zipper, and combinations of thereof The securing element 37 may be deposited on the barrier layer 35, as shown in FIG. 5.

Suitable adhesive that may be used as the securing element 37 may include pressure-sensitive adhesive, which may be applied as continuous or intermittent patches, strips, swirls, or waves, and the like. As used herein, the term pressure-sensitive adhesive refers to any releasable adhesive or releasable tenacious means. Suitable adhesive compositions, include, for example, water-based pressure-sensitive adhesives such as acrylate adhesives; emulsion or solvent-borne adhesives of polyisoprene, styrene-butadiene, or polyacrylate, vinyl acetate copolymer or combinations thereof; hot melt adhesives based on suitable block copoylmers; among other adhesive compositions now known or developed in the future in the art of absorbent article manufacture.

In order to provide sufficient stiffening of the first longitudinally-extending edge 9 during the article manufacturing/packaging process, the layered structure 39 of an absorbent article of the present invention (such as, the absorbent articles 1, 51) includes at least one removable, edge stiffening element 17 (shown in FIGS. 1 through 5). The at least one removable, edge stiffening element 17 may terminate very close to the terminal edge support points 13, 15. The at least one removable, edge stiffening element 17 may be, for example, a release layer, such as, a strip of release paper that is positioned over the securing element 37. For example, for embodiments of the invention in which the securing element 37 includes an adhesive, the at least one removable, edge stiffening element 17 may be a release strip that covers the adhesive.

The at least one removable, edge stiffening element 17 can be formed from any suitable sheet-like material that adheres with sufficient tenacity to the securing element 37 to remain in place prior to use but which can be readily removed when the absorbent article is to be used. Optionally, where adhesive is used as at least part of securing element 37, a coating may be applied to the at least one removable, edge stiffening element 17 to improve the ease of removabilty of the at least one removable, edge stiffening element 17 from the adhesive. Any coating capable of achieving this result may be used, e.g., silicone. Suitable removable, edge stiffening elements 17 are commercially available from, for example, Tekkote of Leonia, N.J. and may have a basis weight of about 40 gsm and a thickness of about 0.051 mm (2 mils), and may be coated with a silicone-based release agent to facilitate removal of the release strip from the adhesive.

The securing element 37 (e.g., positioning adhesive) may be placed in various patterns across the silhouette 3. Similar to the at least one removable edge stiffening element 17, the securing element may have an asymmetric longitudinal disposition with respect to the longitudinally-extending centerline 25.

In one embodiment of the invention, to enhance the stiffness of the first longitudinally-extending edge 9, the securing element 37 is substantially coterminal with the first terminal edge support point 13 and the second terminal edge support point 15. To further enhance the stiffness of the first longitudinally-extending edge 9, the securing element 37 is coterminal with the first terminal edge support point 13 and the second terminal edge support point 15. In another embodiment of the invention, the securing element 37 is substantially coterminal with the entire first longitudinally-extending edge 9. In yet another embodiment of the invention, the securing element 37 is coterminal with the entire first longitudinally-extending edge 9.

In another embodiment of the invention, in order to facilitate securing the absorbent article 1, 51 to the wearer's clothing, the securing element 37 covers a fraction of the silhouette 3 (i.e., a fraction of the area of the silhouette) that is greater than about 90 percent. Note that a high degree of coverage of the securing element 37 may be important for absorbent articles 1, 51 having low stiffness, low flexural resistance, and/or high drapability.

When securing to the absorbent article 1, 51, the removable, edge-stiffening element 17 increases the stiffness of the absorbent article 1, 51. In one embodiment of the invention, the stiffness of the absorbent article 1, 51 without the removable, edge-stiffening element 17 is less than about 20 milligrams (mg), and the stiffness of the absorbent article 1, 51 with the removable, edge-stiffening element 17 is greater than about 30 milligrams (mg). In another embodiment of the invention, the stiffness of the absorbent article 1, 51 without the removable, edge-stiffening element 17 is less than about 15 milligrams (mg) and the stiffness of the absorbent article 1, 51 with the removable, edge-stiffening element 17 is greater than about 35 milligrams (mg).

The stiffness is determined using a Gurley Stiffness Tester (model 4171D) manufactured by Gurley Precision Instruments of Troy, N.Y. “Gurley Stiffness” measures the bendability or flexibility of materials. The lower the Gurley stiffness value, the more flexible the material. The instrument measures the externally applied moment required to produce a given deflection of a test strip of specific dimensions fixed at one end and having a concentrated load applied to the other end. The results are obtained in milligrams. The procedure for determining Gurley Stiffness is as follows:

1. Die cut rectangular samples five samples, each 0.5 inches wide by 1 inch long. The samples should be cut such that the width of the sample is taken along the first longitudinally-extending edge of the absorbent article

2. If release paper is to be removed, eliminate any stickiness that may exist by applying a layer of talcum powder and smoothing out any wrinkles.

3. Calibrate the Gurley tester per manufacturer's instructions.

4. Select a weight that is suitable according to the manufacturers instructions.

5. Position the arm on the Gurley apparatus to the right two inches from the fulcrum.

6. According to the manufacturer's instructions, insert one sample at a time into the clamp and secure sample so the longer edge of the sample is parallel to the clamp.

7. Make sure the pendulum is steady at zero and move the switch to the left so that the entire sample moves across the pendulum.

8. Re-steady the pendulum at zero and move the switch to the right so that the entire sample moves across the pendulum.

9. Record the digital stiffness reading.

10. Repeat steps 1-9 for at least 5 samples for each article to be tested.

11. Average the readings from each article and report the average reading as the stiffness value for the sample being tested.

In one embodiment, the at least one removable, edge stiffening element 17 is substantially coterminal with the first terminal edge-support point 13 and substantially coterminal with the second terminal edge-support point 17. By substantially coterminal it is meant that the at least one removable, edge stiffening element 17 extends to a point that is substantially aligned with (i.e., in vertical registration with) the first terminal edge-support point 13 on the silhouette 3 as well as the second terminal edge-support point 17.

In general, as shown in FIG. 1, respective gaps 85, 87 (measured parallel to the transversely extending centerline 27) may exist between the at least one removable edge stiffening element 17 and the terminal edge support points 13, 17. In one embodiment of the invention, the gaps 85, 87 are less than about 2 mm. In another embodiment of the invention, the gaps are less than about 0.5 mm. In another embodiment of the invention, the at least one removable, edge stiffening element 17 is coterminal with the first terminal edge-support point 13 as well as the second terminal edge-support point 17, i.e., the gaps 85, 87 are essentially zero.

The at least one removable edge-stiffening element 17 is offset from either the second longitudinally-extending edge 11, the first transverse edge 5, second transverse edge 7, or some combination thereof. Referring to FIG. 1, the at least one removable edge-stiffening element 17 extends to an edge 61 that falls within the silhouette 3 of the absorbent article 1. The edge 61 of the at least one removable edge-stiffening element 17 is spaced apart from either the second longitudinally-extending edge 11, the first transverse edge 5, second transverse edge 7, or some combination thereof by a gap 63. The gap 63 is generally large enough such that the at least one removable edge-stiffening element 17 is readily separated from the securing element 37 by a user grabbing the at least one removable edge-stiffening element 17 with her fingers and bending/pulling the at least one removable edge-stiffening element 17. The at least one removable edge-stiffening element 17 is thus releasably affixed to the garment faceable, liquid impervious barrier layer 35. The gap 63 may be greater than about 2.5 mm. In one embodiment of the invention, the gap 63 is greater than about 5 mm. The gap 63 is the greatest linear distance of a line drawn orthogonal to the edge 61, between the edge 61 and a point on any of the edges 5 7, 9, 11, wherein the line traverses a path that is void of any removable edge stiffening element 17.

Note that the edge 61 of the at least one removable edge stiffening element 17 may be linear as depicted in FIGS. 1-4 and 6, the edge 61 may be partially or completely arcuate. For example, portions of the edge 61 may be coterminal with the second longitudinally extending edge 11.

The at least one removable edge stiffening element 17, when divided along the longitudinally-extending centerline 25 may have an asymmetric longitudinal disposition as shown in FIGS. 1-6. In this embodiment of the invention, the at least one removable edge stiffening element 17 is “off-centered” with respect to the longitudinally-extending centerline 25.

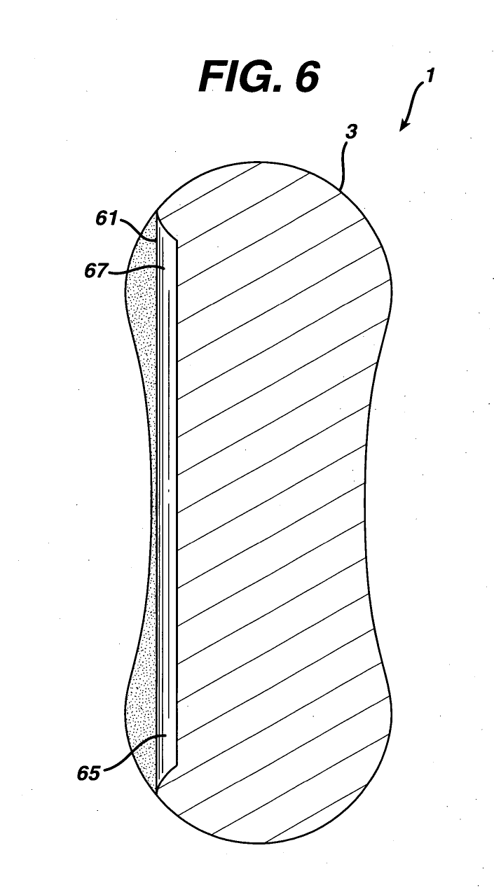

To make the at least one removable edge-stiffening element 17 easy for the user to grab and remove, the at least one removable edge-stiffening element 17 may have a projecting feature 65 as shown in FIG. 6. The projecting feature 65 extends a height above the absorbent article 1, and may be obtained, for example, by folding the at least one removable edge-stiffening element 17 about the edge 61, exposing an underside 67 of the at least one removable edge-stiffening element 17.

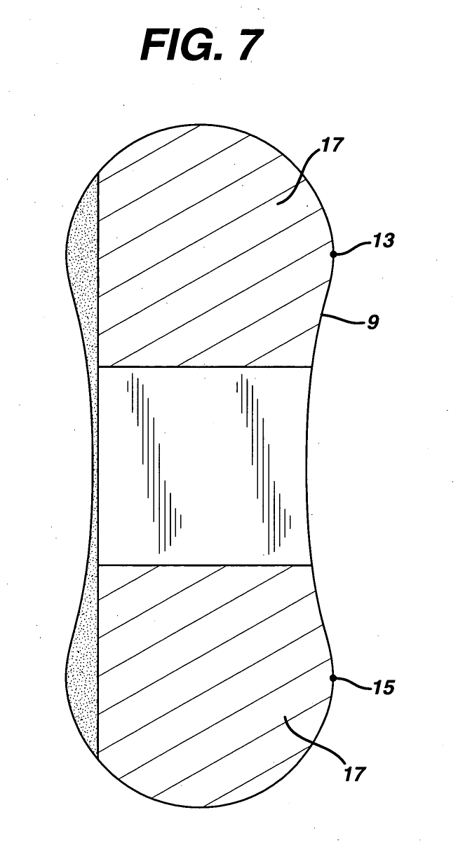

The at least one removable edge-stiffening element 17 is, in one embodiment of the invention, a single removable edge-stiffening element, as shown in FIGS. 1-4 and 6. Alternatively, the absorbent article may include a plurality of removable edge-stiffening elements. For example, the absorbent article 1 having two removable edge-stiffening element 17 is shown in FIG. 7. Furthermore, the at least one removable edge-stiffening element 17 may be substantially coterminal with entire first longitudinally-extending edge 9, as shown in FIGS. 1-4 and 6. Alternatively, the at least one removable edge-stiffening element 17 may be substantially cotermial with only a portion of the first longitudinally-extending edge 9, as shown in FIG. 7.

The absorbent article 1, 51 of the present invention may be characterized as having a low flexural resistance. In particular, the flexural resistance may be less than about 50 grams, as measured without an edge stiffening element 17, e.g., without release paper. In another embodiment of the invention, the absorbent article 1, 51 has a flexural resistance of about 35 grams or less. For a description of absorbent articles having a flexural resistance of about 35 grams or less, as well as a suitable method used to determine flexural resistance of absorbent articles, the reader is referred to currently pending U.S. Ser. No. 10/025,299 entitled, “Drapeable Absorbent Article,” filed on Dec. 19, 2001, commonly owned, and incorporated herein by reference in its entirety.

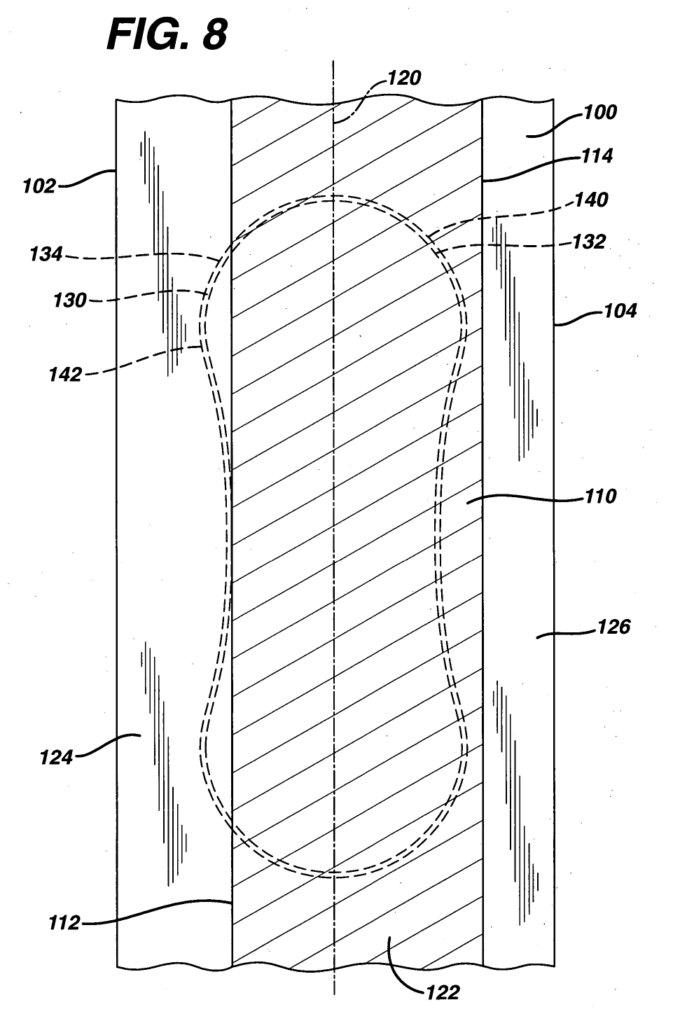

As shown in FIG. 8, absorbent articles of the present invention may be manufactured by providing a web 100 of material along, for example, a conveyer. The web 100 has a directional center 120 and may include one or more material layers selected from, for example, the barrier layer 35, the cover layer 31, and the absorbent system 33. The web 100 has a first edge 102 and a second edge 104 substantially equidistant from the directional center 120. A sheet 110 of edge stiffening material (shown cross-hatched in FIG. 8) is superimposed upon the top surface of the web 100. Typically the web 100 and the sheet 110 are advanced together (in a direction indicated by the arrows of FIG. 8) at a similar speed.

The web 100 and the sheet 110 may be bonded together using, for example, the securing element 37. In one embodiment of the invention, the sheet 110 is treated with the securing element 37 (e.g., coated with adhesive) prior to superimposing the web 100 and the sheet 110. In another embodiment of the invention, adhesive is applied to the web 100 in a pattern such that the sheet 110, when superimposed upon the web 100 will cover the securing element 37.

The sheet 110 is superimposed upon the web 100 such that a first edge 112 of the sheet 110 of the removable, edge stiffening material is registered between the first edge 102 of the web 100 and the second edge 104 of the web 100. A second edge 114 of the web 110 may also be registered between the first edge 102 of the web 100 and the second edge 104 of the web 100, but this is not required.

The superposition of the web 100 and the sheet 110 defines a region of overlap 122 between the web 100 and the sheet 110 as well as a first region 124 that is free of the removable, edge stiffening material, and a region 126 that may be free of removable, edge stiffening material. In one embodiment of the invention, the sheet 110 has an asymmetric disposition with respect to the directional center 120 of the web 100.

The web 100 and the sheet 110 may be sealed in an area 130 between an inner seal boundary 132 and an enclosed trace 134 (both the inner seal boundary 132 and the enclosed trace 134 are shown in phantom in FIG. 8) to bond the various material layers of the webs 100 together. Sealing of the web 100 may be performed by various means known to the art, for example, by advancing the webs 100, 110 between a pair of die rollers, one of which has a raised sealing pattern corresponding to the area 130 formed thereon. Sealing is generally performed at a temperature and pressure sufficient to bond the various material layers of the web 100 together, but the sheet 110 is not bonded to the web 100 to any significant degree.

The web 100 and the sheet 110 are severed along the trace 134 using, for example, another pair of rollers having a pattern of severing edges formed thereon that correspond to the trace 134. Referring again to FIG. 1 as well as FIG. 8, the severing of the webs 100, 110 defines a silhouette, such as the silhouette 3 that includes the first transverse edge 5, the second transverse edge 7 in opposite relation to the first transverse edge, and the first longitudinally-extending edge 9 having the first terminal edge-support point 13 and the second terminal edge-support point 15, the first longitudinally-extending edge 9 opposed to the second longitudinally extending edge 11, the first longitudinally extending edge 9 and the second longitudinally extending edge 11 connecting the first transverse edge 5 and the second transverse edge 7. The first terminal edge-support point 9 and the second terminal edge-support point 13 are substantially within the region of overlap 122 and an edge selected from the group consisting of within the region free of removable, edge stiffening material, extend into the region substantially free of the removable, edge stiffening material. By “substantially within the region of overlap” it is meant that the first terminal edge-support point 9 and the second terminal edge-support point 13 extend no more than 2 mm into the region of overlap 122. In one embodiment of the invention, points 13, 15 extend no more than 0.5 mm into the region of overlap 122. In one embodiment, the points 13, 15 do not extend into the region of overlap 122.

In one embodiment of the invention that is suitable if the enclosed trace 134 is oriented longitudinally with respect to the direction of movement of the webs 100, 110, as shown in FIG. 8, the enclosed trace 134 consists of a segment 140 within the region of overlap and one or more segments 142 within the region free of removable, edge stiffening material, wherein the one or more segments within the region free of removable, edge stiffening material are on one side of the directional center 120 of the web of material.

Upon completing the severing described above, an absorbent article consistent with embodiments of the invention described herein is thereby formed. The process described above differs from the prior processes in that prior processes, the severing may be performed along a trace that is either entirely within the region of overlap, resulting in an absorbent article having release paper that covers the entire backside of the article. Such absorbent articles typically have release paper that is difficult for the wearer to remove. In alternative prior processes, the severing is performed along a trace that extends substantially into regions on either side of the region of overlap, both regions of which are free of release paper. The resulting absorbent article (if the trace is oriented longitudinally with respect to the moving webs and sheets) has release paper that is substantially spaced apart from the longitudinally extending ends. As such, the absorbent article may not have sufficient edge stiffness to facilitate easy packaging/processing. Note that while the enclosed trace 134 is depicted as oriented longitudinally with respect to the direction of movement of the webs 100, 110, in general, the enclosed trace 134 may, alternatively, be oriented in any manner, such as, transversely with respect to the direction of movement of the webs 100, 110.

In order to use the absorbent article 1, 51 of the present invention, the absorbent article is unwrapped from any packaging and the removable edge-stiffening element may be removed by the user. Thereafter the article 1, 51 is placed against the user's body, such as, for example, against the user's pudendal region. The silhouette of absorbent articles of this invention includes those designed to fit garments having conventionally-shaped crotches, e.g., briefs and bikinis as well as abbreviated crotches including thong, string underwear, G-string, Rio cut, Brazilian cut, and the like. As such, the absorbent article 1, 51 of the present invention may be secured to any of these types of clothing, among others.

Any or all of the materials layers, e.g., cover layer, absorbent system, transfer layer, barrier layer, and adhesive layers may be colored. Such coloring includes, but is not limited to, white, black, red, yellow, blue, orange, green, violet, and mixtures thereof. Color may be imparted according the present invention through dying, pigmentation, and printing. Colorants used according the present invention include dyes and inorganic and organic pigments. The dyes include, but are not limited to, anthraquinone dyes (Solvent Red 111, Disperse Violet 1, Solvent Blue 56, and Solvent Green 3), Xanthene dyes (Solvent Green 4, Acid Red 52, Basic Red 1, and Solvent Orange 63), azine dyes (Jet black), and the like.

Inorganic pigments include, but are not limited to, titanium dioxide (white), carbon black (black), iron oxides (red, yellow, and brown), chromium oxide (green), ferric ammonium ferrocyanide (blue), and the like.

Organic pigments include, but are not limited to diarylide yellow AAOA (Pigment Yellow 12), diarylide yellow AAOT (Pigment Yellow 14), phthalocyanine blue (Pigment Blue 15), lithol red (Pigment Red 49:1), Red Lake C (Pigment Red), and the like.

The absorbent article may include other known materials, layers, and additives, such as, foam, net-like material, perfumes, medicaments or pharmaceutical agents, moisturizers, odor control agents, and the like. The absorbent article can optionally be embossed with decorative designs.

The absorbent article may be packaged as unwrapped absorbent articles within a carton, box or bag. The consumer withdraws the ready-to-use article as needed. The absorbent article may also be individually packaged (each absorbent article encased within an overwrap).

From the foregoing description, one skilled in the art can ascertain the essential characteristics of this invention, and without departing from the spirit and scope thereof, can make various changes and modifications. Embodiments set forth by way of illustration are not intended as limitations on the variations possible in practicing the present invention.

What is claimed is:

1. An absorbent article comprising:
A. a silhouette comprising
i. a first transverse edge,
ii. a second transverse edge, wherein the second transverse edge is in opposite relation to the first transverse edge,
iii. a first longitudinally-extending edge having a first terminal edge-support point and a second terminal edge-support point, and
iv. a second longitudinally extending edge, wherein second longitudinally extending edge is in opposite relation to the first longitudinally-extending edge and the first longitudinally extending edge and the second longitudinally extending edge connecting the first transverse edge and the second transverse edge and
B. a layered structure comprising at least one removable, edge-stiffening element wherein the at least one removable edge-stiffening element is offset from an edge selected from the group consisting of the second longitudinally-extending edge, the first transverse edge, the second transverse edge, and combinations thereof, wherein the article has a flexural resistance less than about 50 grams.

2. An absorbent article of claim 1, wherein the removable, edge stiffening element is substantially coterminal with the first terminal edge-support point and substantially coterminal with the second terminal edge-support point.

3. An absorbent article of claim 1, wherein the layered structure further comprises a garment-faceable, liquid impervious barrier layer and the removable, edge stiffening element is releasably affixed to the garment-faceable, liquid impervious barrier layer.

4. An absorbent article of claim 3, wherein the layered structure further comprises an adhesive intermediate the garment-faceable, liquid impervious barrier layer and the removable, edge stiffening element.

5. An absorbent article of claim 4, wherein the adhesive covers at least about 90 percent of the garment-faceable, liquid impervious barrier layer.

6. An absorbent article of claim 4, wherein the adhesive has an asymmetric longitudinal disposition.

7. An absorbent article of claim 1, wherein the at least one removable, edge-stiffening element is offset from the second longitudinally-extending edge a distance greater than about 2.5 millimeters (mm).

8. An absorbent article of claim 1, wherein the at least one removable, edge-stiffening element is offset from the second longitudinally-extending edge a distance greater than about 5 mm.

9. An absorbent article of claim 1, wherein the absorbent article has a thickness between about 0.6 mm and about 2.5 mm.

10. An absorbent article of claim 1, wherein the at least one removable, edge-stiffening element comprises a projecting feature.

11. An absorbent article of claim 1, wherein the layered structure comprises a single removable, edge-stiffening element.

12. An absorbent article of claim 11, wherein the single removable, edge-stiffening element is coterminal with an entire length of the first longitudinally-extending edge.

13. An absorbent article of claim 1, wherein the silhouette is characterized by a longitudinally-extending centerline and the first longitudinally extending edge is non-concave with respect to the longitudinally-extending centerline.

14. An absorbent article of claim 1, wherein the absorbent article has a Gurley stiffness before removing the removable edge stiffening layer that is greater than about 30 milligrams (mg) and a Gurley stiffness after removing the removable edge stiffening layer that is less than about 20 mg.

15. An absorbent article of claim 14, wherein the absorbent article has a Gurley stiffness before removing the removable edge stiffening layer that is greater than about 35 milligrams (mg) and a Gurley stiffness after removing the removable edge stiffening layer that is less than about 15 mg.

16. An absorbent article of claim 4, wherein the adhesive is substantially coterminal with the first terminal edge support point and the second terminal edge support point.

17. An absorbent article of claim 4, wherein the adhesive is coterminal with the first terminal edge support point and the second terminal edge support point.

18. An absorbent article of claim 4, wherein the adhesive is substantially coterminal with the entire length of the first longitudinally-extending edge.

19. An absorbent article of claim 4, wherein the adhesive is coterminal with the entire length of the first longitudinally-extending edge.

20. An absorbent article comprising:
A. a silhouette comprising
i. a first transverse edge
ii. a second transverse edge, wherein the second transverse edge is in opposite relation to the first end, and
iii. a first longitudinally-extending edge having a first terminal edge-support point and a second terminal edge-support point, the first longitudinally-extending edge opposed to a second longitudinally extending edge, the first longitudinally extending edge and the second longitudinally extending edge connecting the first transverse edge and the second end;
B. a layered structure comprising
i. a liquid pervious, body-faceable cover layer;
ii. pervious, garment-faceable barrier layer;
iii. a single removable, edge-stiffening release layer coterminal with the first longitudinally-extending edge, wherein the removable edge-stiffening release layer is offset from the second longitudinally-extending edge a distance greater than about 5 mm; and
iv. a positioning adhesive layer, intermediate the edge-stiffening release layer and the liquid-impervious, garment-faceable barrier layer, wherein the removable, edge-stiffening release layer is releasably adhered to the positioning adhesive layer, and wherein the absorbent article has a flexural resistance less than about 50 grams.

21. A method of forming an absorbent article, wherein the method comprises:
providing a web of material, the web of material having a first edge, a second edge opposed to the first edge, and a directional center between the first edge and the second edge;
superimposing a sheet of edge stiffening material on the web of material such that a first edge of the sheet of removable, edge stiffening material is registered between the first edge of the web of material and the second edge of the web of material, wherein the superposition of the sheet of edge stiffening material and the web of material defines a region of overlap and a region free of removable, edge stiffening material, the region of overlap being adjacent to the region of removable, edge stiffening material; and
severing both the web of material and the web of removable, edge stiffening material superimposed thereupon along a enclosed trace, wherein the enclosed trace defines a silhouette that includes a first transverse edge, a second transverse edge in opposite relation to the first transverse edge, and a first longitudinally-extending edge having a first terminal edge-support point and a second terminal edge-support point, the first longitudinally-extending edge opposed to a second longitudinally extending edge, the first longitudinally extending edge and the second longitudinally extending edge connecting the first transverse edge and the second transverse edge, wherein the first terminal edge-support point and a second terminal edge-support point are substantially within the region of overlap and an edge selected from the group consisting of the second longitudinally-extending edge, the first transverse edge, the second transverse edge, and combinations thereof extend into the region free of removable, edge stiffening material, wherein the article has a flexural resistance less than about 50 grams.

22. A method of claim 21, wherein the web of material comprises a material selected from the group consisting of a body-faceable, fluid pervious cover layer, a garment faceable, liquid-impervious barrier layer, and an absorbent system, and combinations thereof.

23. A method of claim 21, wherein the edge stiffening material is a release paper.

24. A method of claim 21, wherein the sheet of edge stiffening material has an asymmetric disposition with respect to the directional center of the web of material.

25. A method of claim 21, wherein the enclosed trace consists of a segment within the region of overlap and one or more segments within the region free of removable, edge stiffening material, wherein the one or more segments within the region free of removable, edge stiffening material are on one side of the directional center of the web of material.
